(19) United States
(12) Patent Application Publication
(10) Pub. No.: US 2011/0237981 A1
(43) Pub. Date: Sep. 29, 2011

(54) MEDICAL DEVICE AND METHOD FOR OPERATING A MEDICAL DEVICE

(75) Inventor: Axel VOSS, Kreuzlingen (CH)

(73) Assignee: SWITECH MEDICAL AG, Kreuzlingen (CH)

(21) Appl. No.: 13/013,049

(22) Filed: Jan. 25, 2011

Related U.S. Application Data
(60) Provisional application No. 61/297,830, filed on Jan. 25, 2010.

(51) Int. Cl. A61N 7/00

(52) U.S. Cl. 601/2

(57) ABSTRACT

The application pertains to a medical device including a treatment unit for treating patients, an activation device for activating the treatment unit, and an input device for entering an activation code, the input device being coupled to the activation device, and the activation device is arranged to activate the treatment unit for a limited number of treatments and/or for a limited period when a predetermined activation code is entered via the input device.

### RELATED APPLICATIONS

The present invention claims priority to U.S. Provisional Application Ser. No. 61/297,830, filed Jan. 25, 2010, entitled,“KEYCODE ULTASONIC,” the contents of which are hereby expressly incorporated by reference in their entirety.

### TECHNOLOGICAL FIELD

The present invention pertains to a medical device and to a method for operating a medical device, in particular to a medical device for emitting acoustic waves, e.g. for pain management, tissue regeneration or lipolysis and to a method for operating such a device.

### TECHNOLOGICAL BACKGROUND

Medical devices which include a treatment unit for performing treatments on patients, such as the application of acoustic energy to body tissue of a patient, typically include components that undergo an aging process.

Ultrasonic heads which are used for performing lipolysis treatments and/or pressure wave heads for performing tissue regeneration treatments undergo aging processes because of the relatively high acoustic energies generated by these treatment heads. For example, when performing a lipolysis treatment on a patient, an intensity of about 3 W/sqcm (i.e. W/cm²) is applied to the patient in a frequency range of between 0.9 MHz and 4 MHz. Pressure wave devices typically emit high amplitude acoustic waves in single bursts of an energy flux density from 0.01 mJ/sqmm (i.e. mJ/mm²) up to 1.5 mJ/sqmm which cause the electrodes which are exposed to voltages of up to several thousand volts to constantly wear off.

Due to this aging process of the active components of the medical device, the treatment heads emitting ultrasonic acoustic waves or shock waves have to be serviced on a regular basis and have to be replaced from time to time.

Furthermore, some medical devices are not purchased by a customer but are rather rented to the customer. However, it is difficult on the basis of the varying intensity of use by the different customers and the accompanying varying aging processes of some of the components to simply set an average rental rate.

### SUMMARY OF THE INVENTION

It is, thus, an objective of the present invention to provide a medical device as well as a method of operating a medical device which improves the safety of use for the patients.

This objective is solved by means of a medical device with the features of claim 1.

In particular, the medical device includes a treatment unit for treating patients, an activation device for activating the treatment unit and an input device for entering an activation code. The input device is coupled to the activation device and the activation device is arranged to activate the treatment unit for a limited number of treatments and/or for a limited period when a predetermined activation code is entered via the input device.

By the provision of a medical device in which the treatment unit is activated only for a limited number of treatments and/or only for a limited period by means of entering an activation code it can be assured that the treatment unit can only be used for a limited number of treatments or a limited period in which deterioration of any components of the treatment unit can be neglected. Accordingly, the safety for the patient to be treated by means of the treatment unit can be significantly improved because treatment of the patient can only be carried out as long as the treatment unit operates in a safe state.

In a different and/or additional application of the medical device, the medical device may be rented to a medical practitioner on the basis of its actual use. Thus, in order to be in a position to use the treatment unit in order to perform treatments to patients, the respective user (i.e. the medical practitioner) has to enter an activation code into the input device in order to activate, via the activation device, a limited number of treatments and/or a limited treatment period to be performed on the patient. The activation code can be bought, for example, from a service center and/or from the manufacturer of the medical device such that the user pays, in principle, per treatment given to a patient. The advantage of this concept is that the user can adjust the fees to be paid for the right of use of the medical device on a per-use-basis such that the user can afford treating the patients with up-to-date technology.

These advantages are particularly applicable to a medical device including in its treatment unit an ultrasonic and/or pressure wave treatment head for introducing acoustic energy into human tissue. Preferably, the treatment unit includes an ultrasonic head for performing a lipolysis treatment on a patient, preferably emitting ultrasonic waves of an intensity of about 3 W/sqcm and/or at a frequency of between 0.9 and 4 MHz. Preferably, the treatment unit includes a pressure wave head for performing a tissue regeneration treatment, preferably emitting pressure waves of an intensity of about 0.01 mJ/sqmm to 1.5 mJ/sqmm.

The input device may comprise an alphanumerical and/or a numerical keyboard and preferably includes a keyboard, a numeric keypad and/or a touch screen on which the user can enter the activation code. The activation code may be a 10 to 16 digit code.

The activation device may include an analyzer for analyzing the correctness of the activation code entered via the input device in order to activate the treatment unit, the analyzer preferably taking into account a unique system identifier of the medical device, information of a system clock and/or information as to used and unused activation codes stored in a memory. This ensures that the activation code that has been bought by the user is specifically meant for the individual medical device, ensuring that the maximum number of treatments before servicing of the treatment unit becomes necessary again and/or before the treatment unit has to be replaced, is not exceeded. Furthermore, providing an activation code which has to be correlated with a unique system identifier of the medical device reduces the risk that a fraudulently obtained activation code is entered.

The activation device may include a memory for storing unused and/or used activation codes, preferably a storage device for permanently storing the unused and/or used activation codes in the form of a hard disk, a flash memory and/or an optical drive. The unused activation codes are the activation codes which may be used for activating the treatment unit once being correctly entered into the input device. Storing the used activation codes may help the manufacturer of the medical device to track usage of the treatment unit for determining the current service status of the treatment unit and also ensures that the treatment unit cannot be reactivated by simply reentering the identical activation code twice.

The activation device may receive an input from a system clock for recording the elapsed treatment time and correlating it with the activated treatment period, wherein the activation device is set to deactivate the treatment unit as soon as the activated treatment period has been used up. In an alternative or in addition the activation device may include a counter for counting the number of treatments performed and for correlating the number of treatments performed with the activated number of treatments, wherein the activation device is set to deactivate the treatment unit as soon as the activated number of treatments has been used up. By means of these measures it can be assured that the treatment unit is operating only in the contingent of treatments which is allocated by means of the activation code.

The treatment unit may be an ultrasonic unit intended for non-invasive lipolysis, namely for non-invasive disintegration of subcutaneous adipose tissue. In particular, the treatment unit may include a specific ultrasonic transducer for emitting low frequent ultrasonic waves into the adipose tissue. The treatment unit may also be a pressure wave device for performing tissue regeneration treatments which includes a pressure wave applicator for emitting high energy pressure waves. The ultrasonic acoustic energy generates, in the tissue, locally confined degassing effects. These effects are sometimes also referred to as “stable cavitation” effects. The acoustic energy also introduces sheering forces and thermal cell stress which open up the cell walls of the adipocytes and/or induce the release of stored fat from the adipocytes. The contents of the cells, namely triglycerides and fatty acids, are drained into the interstitium and are lymphatically and phagocytically metabolized and eliminated.

By means of the activation of the treatment unit of the medical device it is possible to leverage from the customer fees depending on the actual usage of the medical device. This enables the customer to use up-to-date medical devices without the need to lockup the capital for buying the medical device.

In a specific embodiment, the medical device includes, besides the treatment unit including the ultrasonic transducer and/or the pressure wave applicator and the respective drivers, an input device in the form of a numeric or alphanumeric keyboard, in the form of a touch screen (resistive or capacitive or any other suitable form) which enables the user to enter an activation code. By entering the activation code the user activates, via the activation device, the treatment unit for a limited number of treatments and/or for a limited period.

The activation code preferably is a 10-16 digit activation code which activates the device. The specific number of treatments and/or treatment period for which the treatment unit is activated may be displayed on a display of the medical device. In a preferred embodiment, the already used up treatment units and/or already used up time period of the activated contingent is shown on the display as well.

In case all activated time and/or all activated treatments have been used-up by the user, the treatment unit as such cannot be activated anymore and/or stops (terminates), such that the user cannot perform any further treatments on patients.

Preferably, the user is notified at a specific predetermined time interval and/or number of treatments before it is scheduled that the treatment unit will stop and is prompted to purchase a new activation code to be in a position to continue treatment of patients.

The objective set out above is also solved by means of a method for operating a medical device, including the steps of receiving an activation code entered via an input device and activating via an activation device a treatment unit for performing a treatment on a patient if a predetermined activation code is entered, wherein the treatment unit is activated only for a limited number of treatments and/or only for a limited period.

The method may include the additional step of activating an ultrasonic treatment head for introducing ultrasonic energy into human tissue, preferably an ultrasonic head for performing a lipolysis treatment on a patient, preferably emitting ultrasonic waves of an intensity of about 3 W/sqcm and/or at a frequency of between 0.9 and 4 MHz and/or a pressure wave applicator emitting high intensity pressure waves, preferably of an intensity of about 0.01 mJ/sqmm to 1.5 mJ/sqmm.

The method may include the further step of receiving an input from a system clock, recording the elapsed treatment time and correlating it, in the activation device, with the activated treatment period, wherein the activation device deactivates the treatment unit as soon as the activated treatment period has been used up.

In addition or as an alternative, the method may include the further step of counting the number of treatments performed and correlating, in the activation device, the number of treatments performed with the activated number of treatments, wherein the activation device deactivates the treatment unit as soon as the activated number of treatments has been used up.

The method may include the further step of storing unused and/or used activation codes and assessing, in the activation device, whether the activation code received via the input device corresponds to a used and/or unused activation code and deactivating the treatment unit if the activation code entered corresponds to a used activation code.

The activation code may be purchased via the internet, via telephone, via a mobile phone, via facsimile, via regular mail or via any other suitable distribution channel. The activation code may include the unique identifier of the medical device, in particular its serial number, the date of purchase, the date of expiry as well as the number of purchased therapies and/or the period of treatments. Even though the activation code may include all this information, it may include this information in a coded and/or encrypted manner.

### BRIEF DESCRIPTION OF THE FIGURES

The present disclosure will now be described in greater detail and by way of example only with reference to the attached drawings in which:

FIG. 1 is a schematic overview of a medical device;

FIG. 2 is a schematic flow diagram of a method of operating the medical device; and

FIG. 3 is a schematic flow diagram of the steps of purchasing an activation code.

### DETAILED DESCRIPTION

Exemplary embodiments of a medial device, in particular of an ultrasonic device for lipolysis and/or a pressure wave device for tissue regeneration, as well as of a method for operating the same will now be described in detail with reference to the drawings, wherein like reference numerals identify similar or identical elements and repeated description of these features in the respective embodiments may be omitted to reduce redundancies.

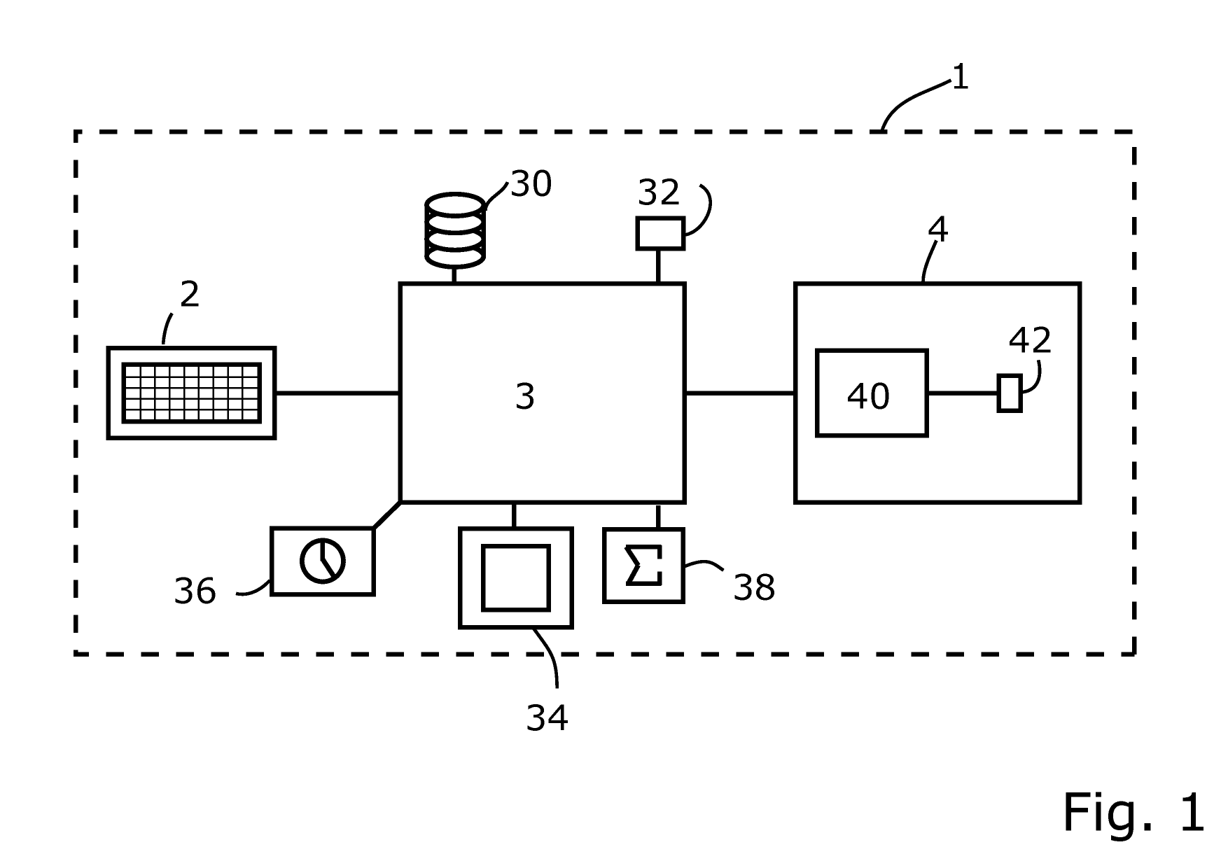

In FIG. 1, a medical device 1 is shown which includes, inter alia, an input device 2 and an activation device 3 which are coupled to one another such that any code entered into the input device 2 is communicated to the activation device 3.

The medical device 1 further includes a treatment unit 4 for treating a patient. The treatment unit 4 includes in the embodiment shown a driver 40 and an acoustic treatment head 42 which is driven by the driver 40. The acoustic treatment head 42 may be suitable for emitting ultrasonic waves into human tissue for performing a treatment, preferably a lipolysis treatment and/or for emitting pressure waves for performing a tissue regeneration treatment. In order to perform a lipolysis or tissue regeneration treatment on a patient, the treatment head 42 is preferably capable of emitting acoustic waves in an intensity of, for example, 3 W/sqcm and/or at a frequency of between 0.9 MHz and 4 MHz and/or pressure waves of an intensity of about 0.01 mJ/sqmm to 1.5 mJ/sqmm.

The input device 2 is shown in the form of a regular keyboard such as a computer keyboard, i.e. an alphanumerical keyboard. However, the input device 2 may also be a numerical keypad, such as the numerical keypads typically used in ATMs, such that a user can easily enter a numerical activation code. The input device could also be a touch screen (capacitive or resistive or any other suitable form) on which the user may enter an activation code. The actual form of the input device 2 is to be determined on the basis of design requirements in the medical device. The input devices already present in the medical device for operating the treatment unit 4 might also be used as the input device.

Other forms of input devices for entering an activation code are also contemplated such as a card reader for reading cards on which an activation code is stored, or any other electronic or optical reading device which enables a user to have some data carrier read out. It is also contemplated that the activation code is input via an input device in the form of an interface to the Internet, a LAN, a WAN, a mobile data standard such as GSM or UMTS or any other data carrier or information carrier which may convey an activation code.

The activation device 3 is set to discriminate between a correct activation code and an incorrect activation code. In case a correct activation code is entered by a user, the activation device 3 activates the treatment unit 4 for a limited number of treatments and/or for a limited period. The activation code may carry an indication as of for how many treatments and/or for how long the treatment unit 4 is activated and can be used. In addition or as an alternative, the activation code activates the treatment unit 4 for a predetermined number of treatments, for example for 12 treatments, and/or for a predetermined period of treatments, for example for a total of 150 minutes of treatment time.

In the activation device 3 used and unused activation codes may be stored in a storage device 30. Storing the used activation codes serves to make sure that an already used activation code cannot be used again to reactivate the treatment unit 4. This ensures that the user uses new activation codes each time the treatment device 4 is activated for a limited number of treatments and/or for a limited period. This enables tracking of the actual usage of the treatment unit 4 by the manufacturer.

In the activation device 3, an activation code entered via the input device 2 may be compared to a unique system identifier of the medical device, in particular to its serial number, which is stored in a memory 32. By the comparison, preferably via an algorithm, it can be assured that only an activation code can be used for activating the treatment unit 4 which is specifically generated for the specific medical device 1. In other words, an activation code preferably is individualized. By this individualization the manufacturer of the medical device 1 may track the usage of the medical device 1 such that the medical device 1 can be serviced and/or replaced depending on the usage. For example, in the case of an acoustic head 42 for lipolysis and/or tissue regeneration which operates at high energies, the acoustic head 42 undergoes an aging process which is dependent on the usage. Accordingly, when the manufacturer of the medical device 1 may track its usage it becomes possible to service or replace the acoustic head 42 in order to maintain performance thereof and, by this, to improve safety for the patients.

The activation device 3 may also analyze the information which might be further embedded in the activation code which is entered via the input device 2 in that it determines whether the activation code has a starting date and/or an expiration date. The activation device 3 may also analyze whether a number of treatments and/or a period is specified by the activation code for which the treatment unit 4 may be activated.

The results of this analysis may be displayed on a display 34 showing the overall number of treatments and/or the overall treatment period activated by means of the activation code. It may also show the number of treatments and/or treatment period already used such that a user is clearly informed as to the number of treatments and/or treatment time which is still available on the basis of the recently entered activation code.

The activation device 3 may be coupled to an internal clock 36 of the medical device 1 which supplies to the activation device not only the current time and date but also enables the activation device 3 to correctly measure the elapsed treatment time when the treatment unit 4 is activated and is actually used. A counter 38 may also be present in order to count the number of treatments performed with the treatment unit 4.

A “treatment” is to be understood in the sense that a patient receives a full treatment, independent of the time elapsed. In the case of a lipolysis treatment, a treatment is a full treatment given to the patient in one treatment session, i.e. from the first application of acoustic waves to the last application of acoustic waves.

After the number of treatments and/or the treatment period has elapsed, the activation device 3 shuts down the treatment unit 4 and prompts the user to enter a new activation code.

In a specific embodiment, the medical device 1 includes, besides the treatment unit 4 including the acoustic transducer 42 and the respective driver 40, an input device 2 in the form of a numeric or alphanumeric keyboard, in the form of a touch screen (resistive or capacitive or any other suitable embodiment) which enables the user to enter an activation code. By entering the activation code the user activates, via the activation device 3, the treatment unit 4 for a limited number of treatments and/or for a limited period.

Preferably, the activation code is a 10-16 digit activation code which activates the device. The specific number of treatments and/or treatment period for which the treatment unit 4 is activated may be displayed on a display 34 of the medical device 1. In a preferred embodiment, the already used up treatment units and/or already used up time period of the activated contingent is shown on the display 34 as well.

In case all activated time and/or all activated treatments have been used-up by the user, the treatment unit 4 as such cannot be activated anymore and stops, such that the user cannot perform anymore treatments on patients.

Preferably, the user is notified at a specific predetermined time interval and/or number of treatments before it is scheduled that the treatment unit 4 will stop and is prompted to purchase a new activation code to be in a position to continue treatment of patients.

The treatment unit 4 may be an ultrasonic unit intended for non-invasive lipolysis, namely for non-invasive disintegration of subcutaneous adipose tissue and/or a pressure wave applicator for performing a tissue regeneration treatment. In particular, the treatment unit 4 may include a specific acoustic transducer 42 for emitting low frequent ultrasonic waves into the adipose tissue or single burst high energy pressure waves into any mammal tissue. In the tissue, e.g. the ultrasonic acoustic energy generates locally confined degassing effects, sometimes referred to as stable cavitation, and introduces sheering forces and thermal cell stress which open up the cell walls of the adipocytes and/or induce the release of fat from the adipocytes. The contents of the cells, namely triglycerides and fatty acids, are drained into the interstitium and are lymphatically and phagocytically metabolized and eliminated.

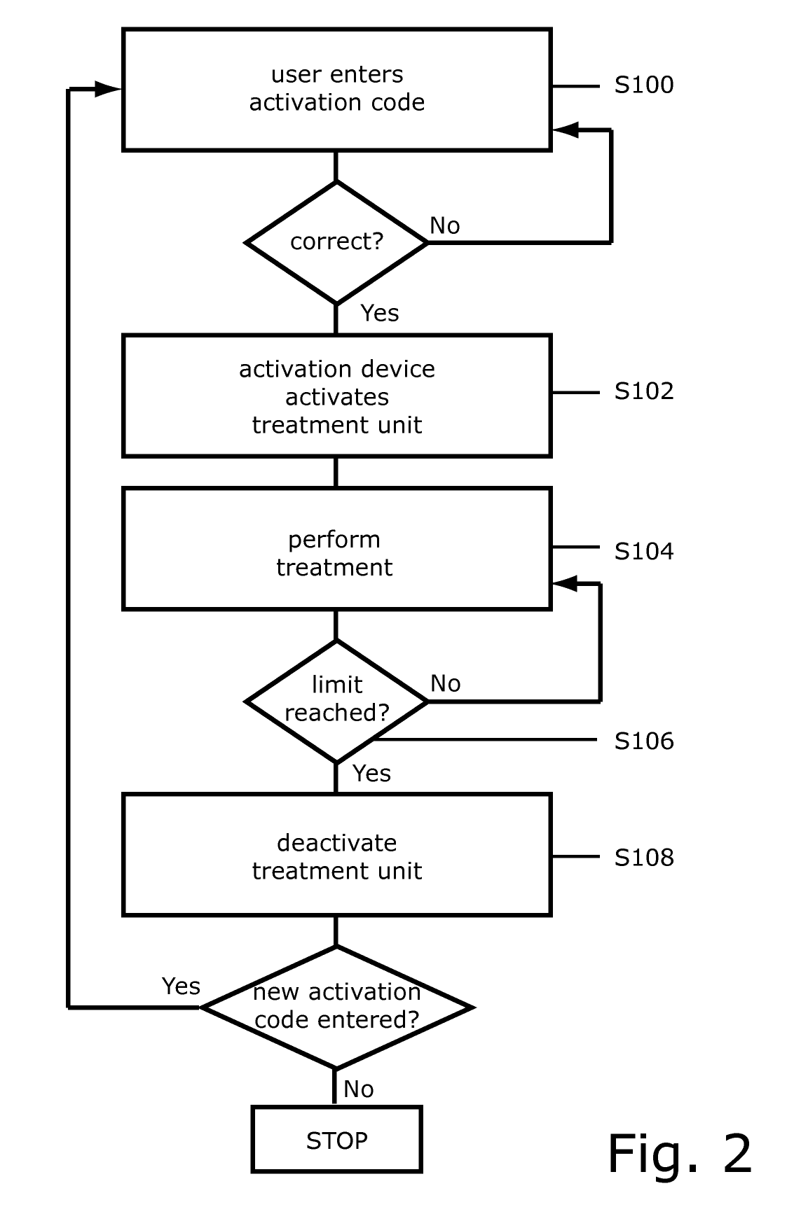

FIG. 2 shows schematically a method of operating the medical device 1 described with respect to the embodiment shown in FIG. 1 above.

In particular, in step S100 the customer enters an activation code into the input device.

If the activation code is not correct, the customer may be prompted to enter another activation code. In a preferred embodiment, after a few erroneous activation codes have been entered, the activation device is blocked. For example after three or five or seven times entering a wrong activation code, the activation device is blocked and can only be unblocked by a service technician or by the manufacturer.

If the activation code entered is correct, the activation unit activates the treatment unit for treatment in step S102. Accordingly, a treatment can be performed carried out in step S104.

During treatment, it is constantly checked whether the time limit has been reached. If the time limit has not been reached in step S106, treatment may be continued. If the time limit has been reached, the treatment unit is deactivated in step S108.

In a different embodiment, it is not the time that is constantly monitored, but it is the number of treatments that is monitored. The activation device constantly counts the number of treatments performed by the treatment unit and if the maximum number of treatments has been reached, the activation unit will not allow activation of the treatment unit any more and the treatment unit will be deactivated.

After the treatment unit has been deactivated, the user is prompted to enter a new activation code. If a new activation code is entered and the activation code is correct, treatment can be continued until the new limit is reached. If no new activation code is entered, the medical device will stop.

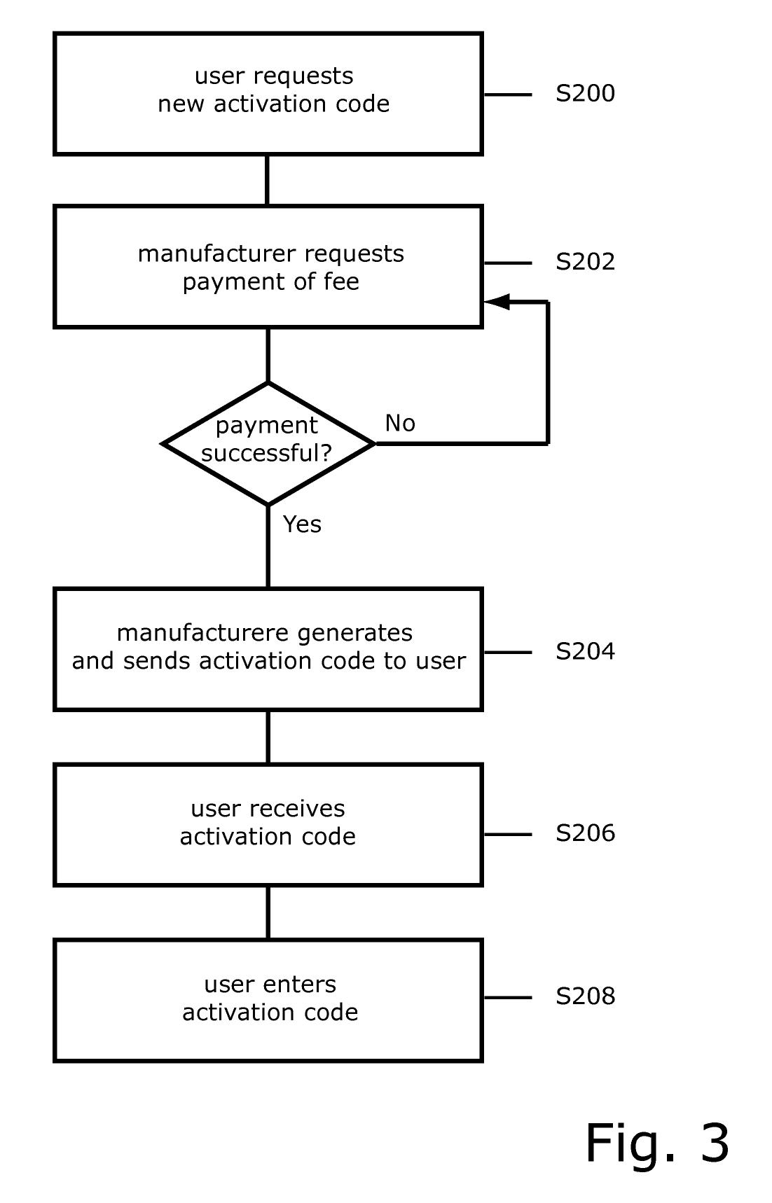

FIG. 3 schematically shows a process of purchasing a new activation code.

In order to purchase a new activation code, the user requests in step S200 a new activation code from a service center and/or from the manufacturer or any other suitable instance.

The service center and/or the manufacturer requests payment of a fee from the user in step S202. If the payment is successful, the manufacturer generates and sends an activation code to the user in step S204.

If payment is not successful, the service center and/or manufacturer will request from the user the payment of the fee again.

After the user has received the activation code in step S206 the user may enter the activation code into the input device in step S208. From here, the method may continue at step S100 of FIG. 2, for example.

The activation code can preferably be bought by means of electronic payment methods, for example via a credit card, via bank transfer and/or any other suitable electronic payment methods.

Preferably, the user receives, after having purchased the treatments or treatment time, an e-mail or any other suitable communications with which the activation code is communicated to the user.

As the activation code is specific for the medical device of the user as it includes the unique identifier of the medical device and, in particular, the serial number of the medical device, improved measures as to the security of transmission of the activation code is not necessary. However, it is preferred to transmit the activation code over a secure socket layer server or any other suitable means for secure transmission.

1. Medical device including:
a treatment unit for treating patients;
an activation device for activating the treatment unit; and
an input device for entering an activation code, the input device being coupled to the activation device, wherein the activation device is arranged to activate the treatment unit for a limited number of treatments and/or for a limited period when a predetermined activation code is entered via the input device.

2. Medical device according to claim 1, wherein the treatment unit includes an ultrasonic treatment head for introducing ultrasonic energy into human tissue, the ultrasonic head configured for performing a lipolysis treatment on a patient by emitting ultrasonic waves of an intensity of about 3 W/sqcm and/or at a frequency of between 0.9 and 4 MHz.

3. Medical device according to claim 1, wherein the treatment unit includes a pressure wave treatment head for introducing pressure wave energy into human tissue, the pressure wave head configured for performing a tissue regeneration treatment on a patient by emitting high energy pressure waves, preferably of an intensity of about 0.01 mJ/sqmm to 1.5 mJ/sqmm.

4. Medical device according to claim 1, wherein the input device comprises an alphanumerical and/or a numerical keyboard, a numeric keypad and/or a touch screen.

5. Medical device according to claim 1, wherein the activation device is arranged to receive, via the input device, a 10 to 16 digit activation code.

6. Medical device according to claim 1, wherein the activation device includes an analyzer for analyzing the correctness of the activation code entered via the input device in order to activate the treatment unit, the analyzer taking into account a unique system identifier of the medical device, information of the system clock and/or information as to used and unused activation codes stored in a memory.

7. Medical device according to claim 1, wherein the activation device includes a storage device for storing unused and/or used activation codes,the storage device for permanently storing the unused and/or used activation codes such as a hard disk, a memory, a flash memory and/or an optical drive.

8. Medical device according to claim 1, wherein the activation device receives an input from a system clock for recording the elapsed treatment time and correlating it with the activated treatment period, wherein the activation device is set to deactivate the treatment unit as soon as the activated treatment period has been used up.

9. Medical device according to claim 1, wherein the activation device includes a counter for counting the number of treatments performed and for correlating the number of treatments performed with the activated number of treatments, wherein the activation device is set to deactivate the treatment unit as soon as the activated number of treatments has been used up.

10. Medical device according to claim 1, wherein the activation device communicates with the user via a display.

11. Method for operating a medical device, including the steps of:
receiving an activation code entered via an input device;
activating via an activation device a treatment unit for performing a treatment on a patient if a predetermined activation code is entered, wherein the treatment unit is activated for a limited number of treatments and/or for a limited period only.

12. Method according to claim 11, including the additional step of activating an ultrasonic treatment head for introducing ultrasonic energy into human tissue, the ultrasonic head configured for performing a lipolysis treatment on a patient, by emitting ultrasonic waves of an intensity of about 3 W/cm² and/or at a frequency of between 0.9 and 4 MHz.

13. Method according to claim 11, including the additional step of activating a pressure wave treatment head for introducing pressure wave energy into human tissue, the pressure wave head configured for performing a tissue regeneration treatment on a patient, by emitting high intensity pressure waves, preferably of an intensity of about 0.01 mJ/sqmm to 1.5 mJ/sqmm.

14. Method according to claim 11, wherein the activation code received is a 10 to 16 digit activation code.

15. Method according to claim 11, including the further step of receiving an input from a system clock, recording the elapsed treatment time and correlating it, in the activation device, with the activated treatment period, wherein the activation device deactivates the treatment unit as soon as the activated treatment period has been used up.

16. Method according to claim 11, including the further step of counting the number of treatments performed and correlating, in the activation device, the number of treatments performed with the activated number of treatments, wherein the activation device deactivates the treatment unit as soon as the activated number of treatments has been used up.

17. Method according to claim 11, including the further step of storing unused and/or used activation codes and assessing, in the activation device, whether the activation code received via the input device corresponds to a used and/or unused activation code and deactivating the treatment unit if the activation code entered corresponds to a used activation code.
